(19) United States
(12) Patent Application Publication
(10) Pub. No.: US 2025/0113794 A1
(43) Pub. Date: Apr. 10, 2025

(54) METHOD OF USING/APPLYING A KERATIN HYDROLYSIS PEPTIDE SOLUTION TO ENHANCE THE YIELD AND QUALITY OF CUCUMBER FRUITS

(71) Applicant: CH Biotech R&D Co., Ltd., Nantou City (TW)

(72) Inventors: Kai XIA, Nantou (TW); Hsin-Yu CHEN, Kaohsiung City (TW); Jie-Chao YOU, New Taipei City (TW); Nai-Hua YE, Changhua (TW)

(73) Assignee: CH Biotech R&D Co., Ltd., Nantou City (TW)

(21) Appl. No.: 18/403,246

(22) Filed: Jan. 3, 2024

(30) Foreign Application Priority Data
Oct. 6, 2023 (TW) 112138603

(51) Int. Cl. A01H 3/04; C07K 1/12; C07K 14/47; G01N 33/68

(52) U.S. Cl. CPC A01H 3/04; C07K 1/12; C07K 14/4741; G01N 33/6848; G01N 2333/4742

(57) ABSTRACT

Present invention teaches the method of using a keratin hydrolysis peptide (“KHP”) solution to enhance production yield and fruit quality of cucumber by selectively choosing specific weights of feathers and water, and treating the mixture to a high-temperature high-pressure hydrolysis process, the resulting solution is confirmed to contain at least 253 peptides and, at early growth stage, applied to the soil around and/or leaf surface of the cucumber plants. Optionally, the KHP solution can be diluted by water, as disclosed in the specification, before administering as taught herein.

### PRIORITY CLAIM TO FOREIGN APPLICATION

Applicant hereby makes priority claim to a Taiwan application, number 112138603, having the Taiwan filing date of Oct. 6, 2023.

### BACKGROUND OF THE INVENTION

Present invention disclosed and claimed the method and application of a keratin hydrolysis peptide (“KHP”) solution to cucumber plants, in order to promote better growth and yield of the cucumber fruits

The KHP solution is made by a hydrolysis process based upon a mixture of feathers and water, at different weight ratio, via a high-temperature and high-pressure process, resulting in a solution that has many beneficial applications in the fields of horticulture, agriculture and potentially other farming businesses.

Hydrolyzed keratin has long been used to strengthen hairs, reduce breakage, and minimize damages. Some form of chemical process is used to break down the large protein molecule of the hydrolyzed keratin so that it can be better absorbed and utilized to benefit the hair and other tissues.

Such hydrolyzed keratin solution is also known to be applied to crops, in the hope of promoting the plants' health and increasing production yield by different methods of applications. Some form of chemical process was attempted, and used, to break down the large protein molecule of the hydrolyzed keratin so that it can used in the intended direction/purpose. The chemical process, though, is generally not favored in the agricultural industry.

In the ever increasingly urgent context of global climate warming, the average temperature in Taiwan has risen roughly 1.4° C. over the past 100 years. This temperature rise, especially in summer times where the 35° C. and above temperature reading happens all the time, greatly affected the growth of cucumber plants, both in the health and the production yield by weight. This is so despite many farms already adopted some form of “tunnel greenhouse” and other covered formed of indoor planting mechanism to avoid the scorching sun from directly heating the plants and the surrounding air/soil. Some farms even installed blowing fans to reduce the heat, with little observable help.

Studies have shown that, under a higher-temperature environment, the main roots of cucumber plants become thinner and fibrous, reducing its effectiveness in taking in the nutrients in the soil. When the temperature reaches 42° C., there is no more growth of the roots for certain specific cucumber species; this certainly stunned the nutrient intake for the plants.

At higher temperature, it is also found that the capacity of the plants' photosynthesis is reduced, leading the less nutrients being produced by the cucumber plants, as well as lower flowering rates and fruiting rates. For some fruits that are already taking shape, higher rate of deformation and fruit abortion is observed, resulting in the health problems and the net production yield of the cucumber fruits.

To counter the problems, the present invention used the hydrolyzed keratin and applied to young cucumber plants, by infusing into the soil around the plants, spraying to the plants and specifically spraying to the leaf surface of the plant, to promote healthier growth and increase production yield.

By the inventors' actual tests in the field, it was proved that the use of the KHP solution enhanced the growth of leaf surface and the content of chlorophyll, thus added to the capacity of the plants' photosynthesis function, and ultimately resulting in the better health of the cucumber plants and production yield.

### SUMMARY OF THE INVENTION

Given the known problems as stated above, there are existing technologies that can be used to counter the adversary conditions described above by resorting to a solution made by keratin, and not by using fertilizers that pose inherent risks to the environment.

It is known that feather contains 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of α-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

Nevertheless, there is no reported successful case or study to show that KHP solutions can be relied upon to help with the healthy growing and production of cucumber plants.

The inventors of present application, under the aegis of CH Biotech, developed a method of creating a keratin solution and a method of applying the solution to the cucumber plants. The inventors proved by experiments that such methods in fact helped with the increase of cucumber fruit's health and production.

In an embodiment method of the KHP solution to promote the production yield and quality of the cucumber fruits, consisting the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm;
d. applying the solution to the soil around cucumber plants at early growth stage.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The method of using a keratin hydrolysis peptide (KHP) solution stated above where the solution is sprayed to the cucumber plants at early growth stage; alternatively, the solution is sprayed to the leaf surface of the cucumber plants.

The method of using a keratin hydrolysis peptide (KHP) solution stated above where the solution is diluted with water by volume at the ratio of 1:100-1000 and sprayed to the cucumber plants at the early growth stage.

The method of using a keratin hydrolysis peptide (KHP) solution stated above where the solution is diluted with water by volume at the ratio of 1:300-500 and is sprayed to the leaf surface of the cucumber plants.

The inventors of present application conducted field test on cucumber plants and confirmed the effectiveness of the invention disclosed and claimed herein.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

FIGS. 1A and 1B show the chlorophyll count by SPAD 502 Plus instrument by the measurements taken from the leaves of 20 cucumber plants in the check (CK) group and in the KHP solution group. The A and B denote two separate farming fields planted with the cucumber plants.

FIGS. 2A and 2B show the leaf length measurements taken from 20 cucumber plants in the check (CK) group and in the KHP solution group. The A and B denotes two separate farming fields planted with the cucumber plants.

FIGS. 3A and 3B show the cucumber fruit counts taken from 20 cucumber plants in the check (CK) group and in the KHP solution group. The A and B denote two separate farming fields planted with the cucumber plants.

FIG. 4 shows the amount of aborted cucumber fruits in the B field, within a 25-day period, for the check (CK) group and the KHP group.

FIGS. 5A and 5B show the water content measurement taken from 20 cucumber plants in the check (CK) group and in the KHP solution group. A and B denote two separate farming fields.

### DETAILED DESCRIPTION OF THE INVENTION

The keratin hydrolysis peptide (“KHP”) solution of present invention has two embodiments based upon the weight ratio between feathers and water. One embodiment uses 66 kg of feathers mixed with 44 kg of water; the other embodiment uses 50 kg of feathers mixed with 40 kg of water. The feathers have a water content of 50%.

The mixture then is treated by a high-temperature and high-pressure process as further described below.

In a first embodiment method of the KHP solution used on cucumber plants to promote the production yield and quality of the cucumber fruits, consisting the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜ 4.5×10⁵ ppm;
d. applying the solution to the soil around cucumber plants at early growth stage.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The method of using a keratin hydrolysis peptide (KHP) solution stated above where the solution is sprayed to the cucumber plants at early growth stage.

The method of using a keratin hydrolysis peptide (KHP) solution stated above where the solution is sprayed to the leaf surface of the cucumber plants, at selected intervals such as weekly spraying.

The method of using a keratin hydrolysis peptide (KHP) solution stated above where the solution is diluted with water by volume at the ratio of 1:100-1000 and is sprayed to the cucumber plants at the early growth stage.

The method of using a keratin hydrolysis peptide (KHP) solution stated above where the solution is diluted with water by volume at the ratio of 1:300-500 and is sprayed to the leaf surface of the cucumber plants.

The two embodiments' hydrolysis parameters are shown in the table herein; though the claims and field tests are focused on first embodiment (the solution is referred to as KHP-1 solution)

| | | Water | | | | | | |
|---|---|---|---|---|---|---|---|---|
| | | Content | | | | | | |
| Feather | Water | in | | | | Product | | |
| Weight | Weight | Feathers | Pressure | Temp | Time | Weight | Mass | Concentr. |
| (kg) | (kg) | % | (kg/cm²) | (° C.) | (min) | (kg) | (Dalton) | (ppm) |
| 66 | 44 | 50% | 16 | 195 | 40 | 131.65 | 593.3~3828.0 | 200000 |
| 50 | 40 | 50% | 12 | 185 | 80 | 136.66 | 593.3~3508.9 | 301500 |

To prove the effectiveness of the KHP solution application to cucumber plants, the inventors conducted tests in two separate fields, identified as field A and field B, planting LuGuang species and ChuanFu species respectively.

Starting 2 weeks after seeding the cucumber plants, the leaf surface would be sprayed with the KHP solution (choosing only the first embodiment, KHP-1, the 66 kg feather and 44 kg water combination) once a week, for 4 weeks. A check group (identified as CK) is only sprayed with water during the same period of time.

The KHP-1 solution is diluted 500 times by water (volume); 20 cucumber plants from each field A and B are then selected to test for various measurements.

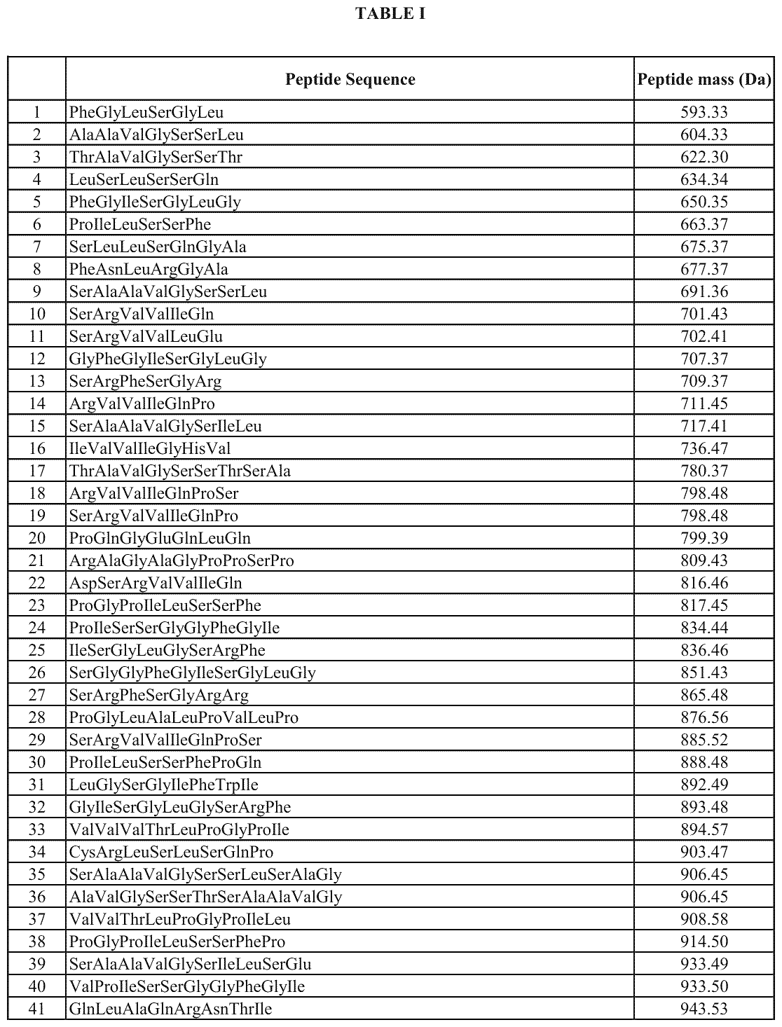

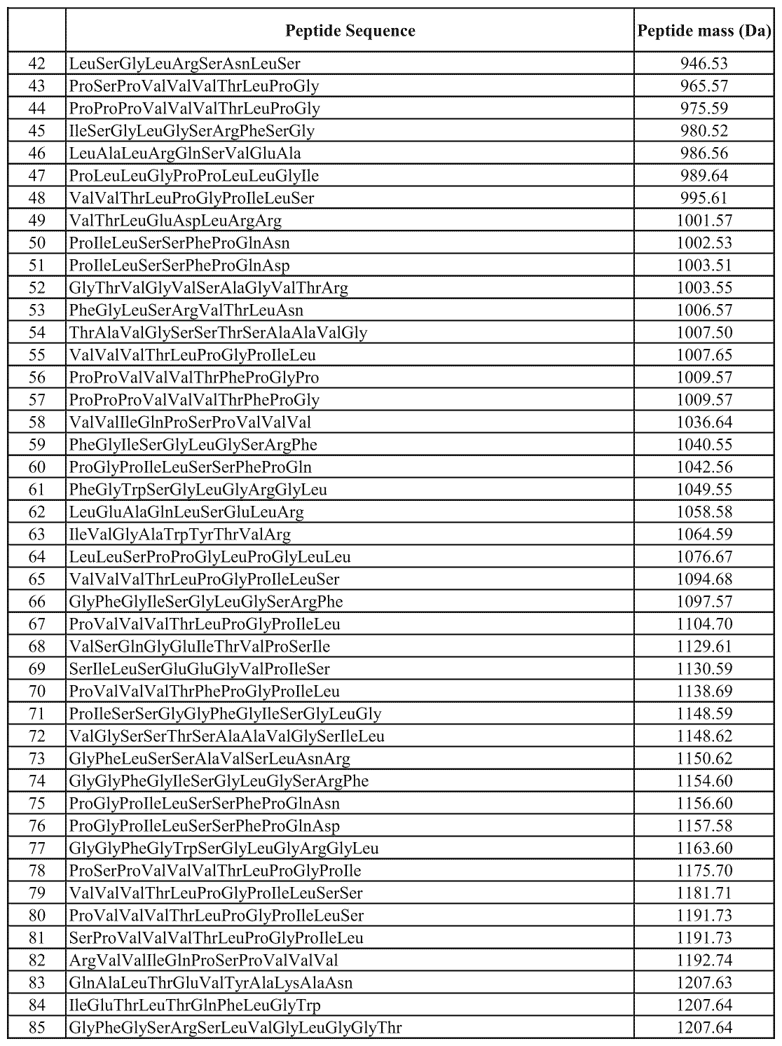

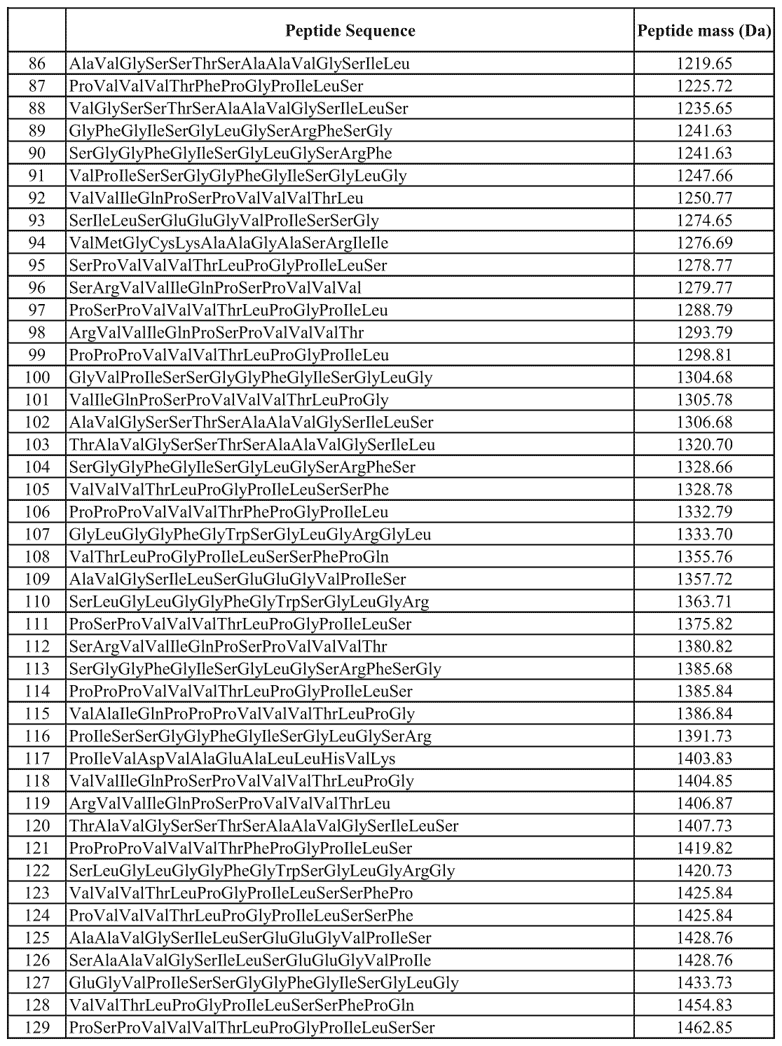

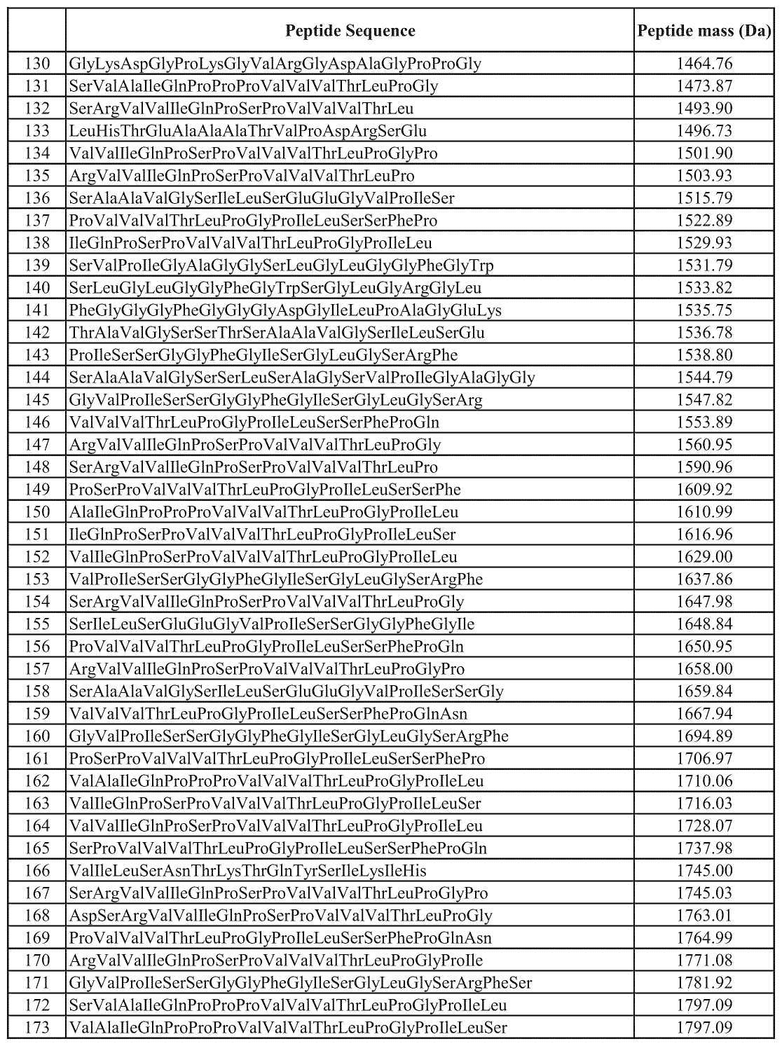

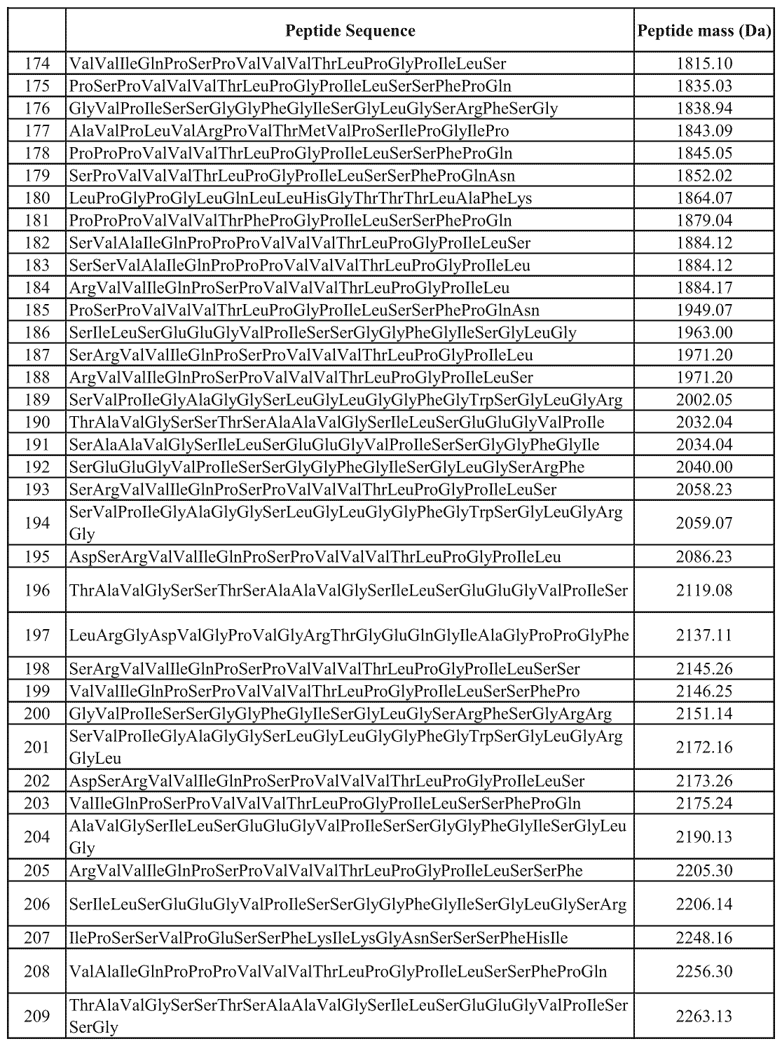

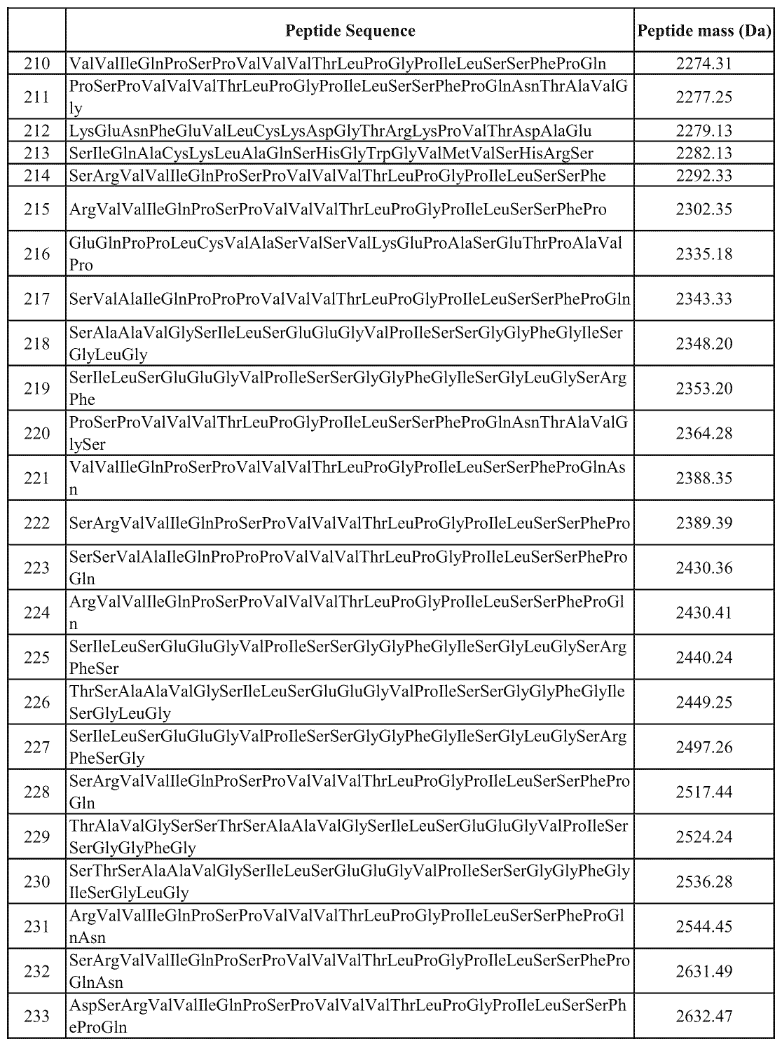

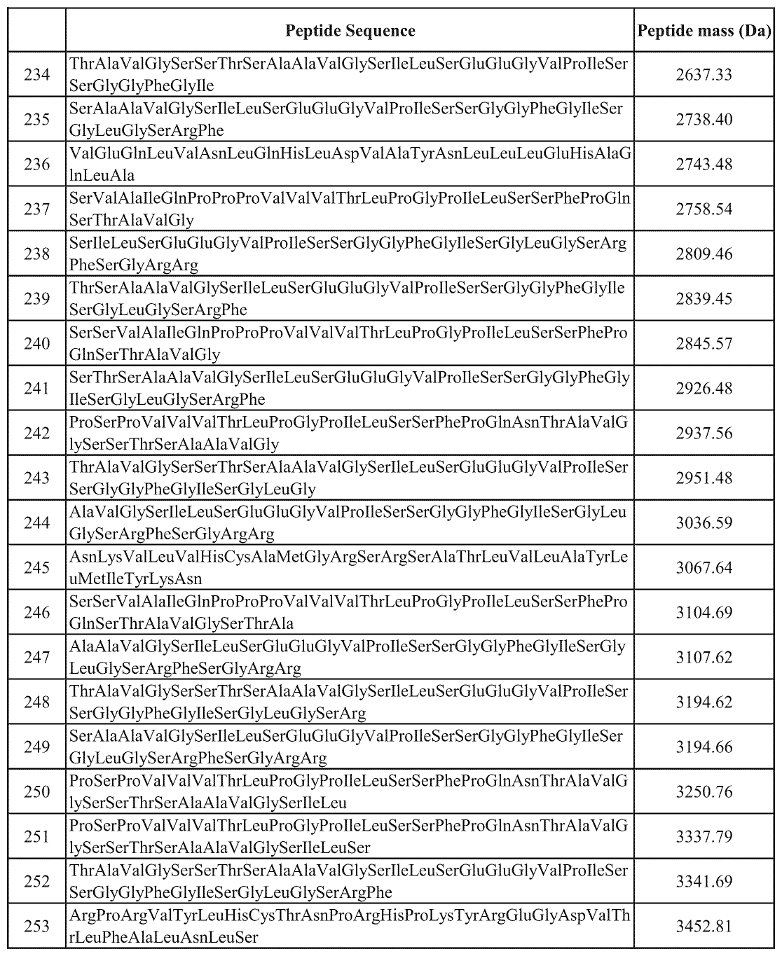

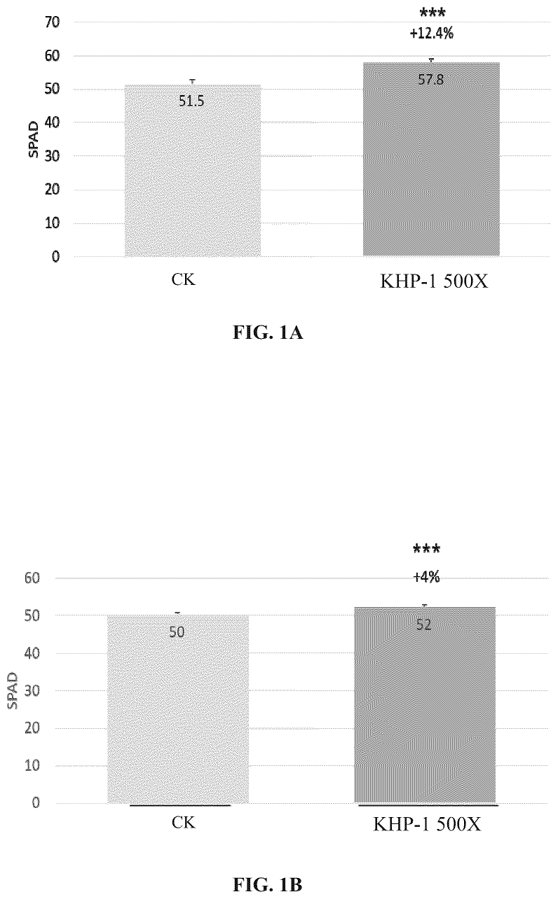

As shown in FIGS. 1A and 1B, the chlorophyll count by the measurements taken from the leaves of 20 cucumber plants in the check (CK) group and in the KHP-1 solution group (using the SPAD 502 Plus instrument), there is a 12.4% increase of by the KHP-1 group over the CK group in field A and the leaves have a color of deep green, a higher count of chlorophyll even by visual inspection. In field B, the KHP-1 group's chlorophyll reading is higher than CK group by 4%.

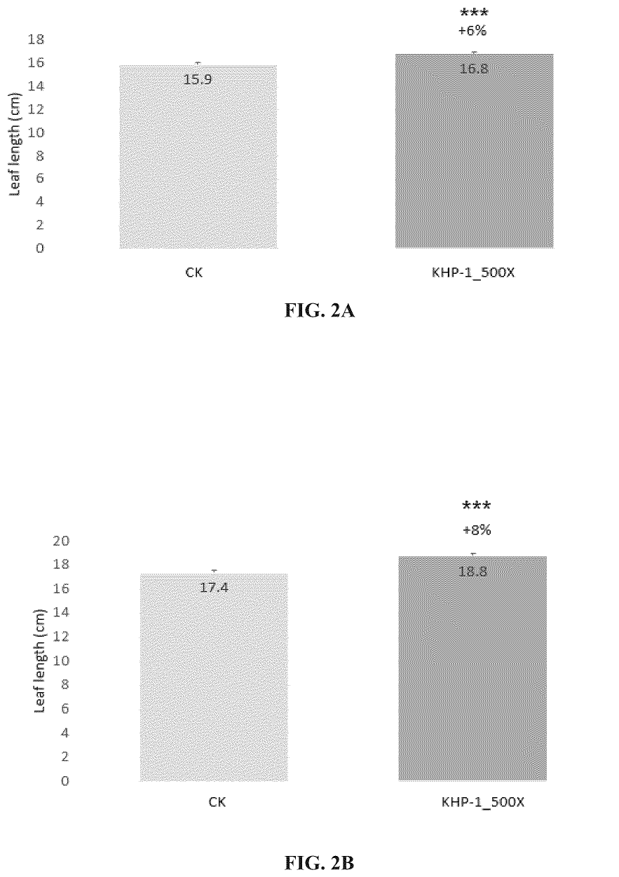

FIGS. 2A and 2B show the leaf length measurements taken from 20 cucumber plants in the check (CK) group and in the KHP-1 solution group. The leaf length of KHP-1 group in field A is about 6% longer than that of the CK group when sprayed with KHP-1 solution. The leaf length in field B is about 8% longer. This shows that the leaf area has substantial increase when sprayed with the KHP-1 solution, at a dilution rate (by volume) of 500 times.

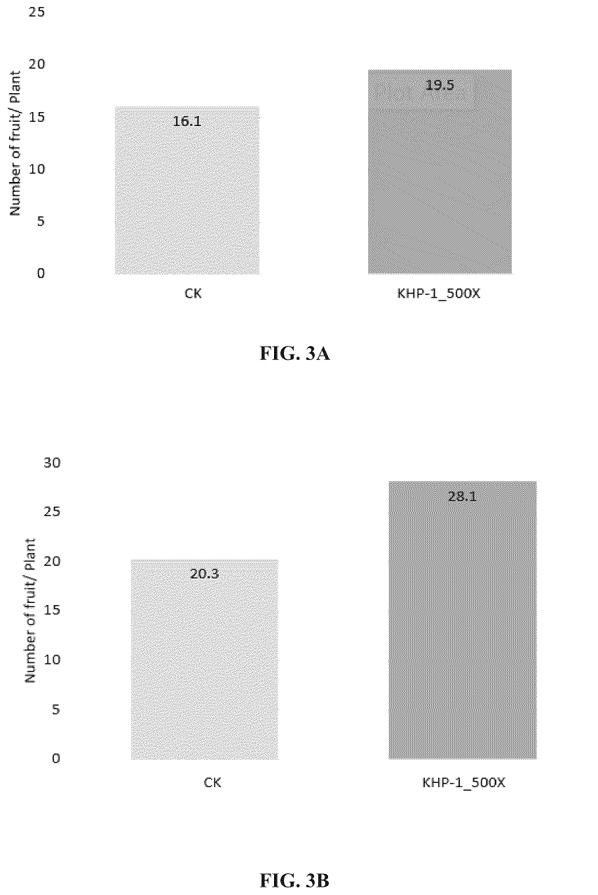

In the tests conducted, the inventors harvested cucumber fruits, and eliminated those that are shorter than 15 CM, with a curvature larger than 3 CM, and selected those weight between 80 and 170 grams. The per plant production counts (average from the 20 plants in field A and field B) are calculated and noted in FIGS. 3A and 3B. As shown, the production counts in the KHP-1 group are substantially higher than the counts in CK group.

Cucumber fruits may become aborted when the flowering stage is not complete and the fruit becomes yellow, showing signs of stunted growth. The inventors conducted tests, collected the aborted cucumber fruits from 48 plants and tallied up the aborted fruit counts. Field A's aborted fruit counts are shown in the table below (the first and second counts are 6-days apart).

| | CK | KHP-1 |
|---|---|---|
| First Aborted Count | 195 | 142 |
| Second Aborted Count | 183 | 91 |
| Total | 378 | 233 |

Field B's aborted fruit counts are shown in FIG. 4. The tests, done in a 25-day duration, show that KHP-1 group (in both fields A and B) have substantially lower amount of aborted fruit count than the CK group.

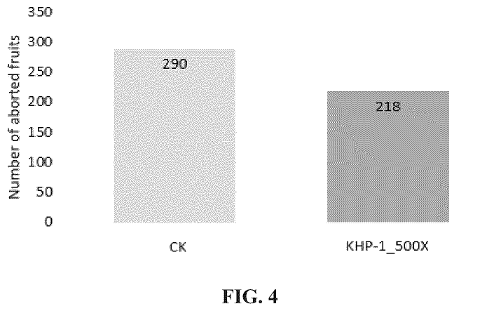

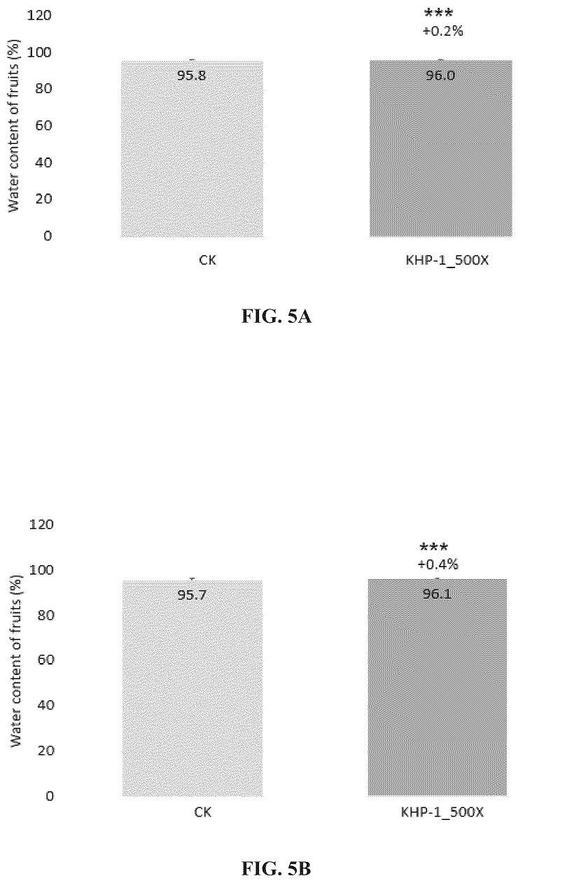

FIGS. 5A and 5B show the water content measurement taken from 20 cucumber fruits in the check (CK) group and in the KHP-1 solution group, for both fields A and B. The harvested fruits are weighed, then are subjected to a drying process by which the water content is then measured. The results are noted in Figs A and B, showing substantially higher water content in KHP-1 group than in CK group.

The experiments done, in accordance with the method disclosed herein, proved that the cucumber plants, when applied with the KHP-1 solution, at the various dilution ratios, attain substantially better growth results than the check group where only water and normal nutrients were given under the same conditions.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to enhance the production yield and quality of cucumber fruits, comprising the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜ 4.5 ×10⁵ ppm;
d. applying the solution to soil around cucumber plants at early growth stage.

2. The method of using a keratin hydrolysis peptide (KHP) solution of claim 1 where the solution is sprayed to the cucumber plants at early growth stage.

3. The method of using a keratin hydrolysis peptide (KHP) solution of claim 1 where the solution is sprayed to leaf surface of the cucumber plants.

4. The method of using a keratin hydrolysis peptide (KHP) solution of claim 1 where the solution is diluted with water by volume at the ratio of 1:100-1000 and is sprayed to the cucumber plants at the early growth stage.

5. The method of using a keratin hydrolysis peptide (KHP) solution of claim 1 where the solution is diluted with water by volume at the ratio of 1:300-500 and is sprayed to the leaf surface of the cucumber plants.
